(19) United States
(12) Patent Application Publication
(10) Pub. No.: US 2011/0177166 A1
(43) Pub. Date: Jul. 21, 2011

(54) Galenical Formulation Comprising Aliskiren and Process for its Preparation by Melt Extrusion Granulation

(76) Inventors: Indrajit Ghosh, Parsippany, NJ (US); James Kowalski, Belle Mead, NJ (US); Jennifer Snyder, Morris Plains, NJ (US); Wei-Qin Tong, Basking Ridge, NJ (US); Sudha Vippagunta, Morris Plains, NJ (US)

(21) Appl. No.: 13/120,182

(22) PCT Filed: Sep. 23, 2009
(86) PCT No.: PCT/US09/57993

§ 371 Date: Mar. 22, 2011

Related U.S. Application Data
(60) Provisional application No. 61/161,799, filed on Mar. 20, 2009.
(60) Provisional application No. 61/099,595, filed on Sep. 24, 2008.

(51) Int. Cl. A61K 31/41; A61K 31/165; A61K 9/20; A61P 9/12; A61P 9/00; A61P 3/10; B29B 9/02

(52) U.S. Cl. 424/465; 514/616; 514/381; 264/141

(57) ABSTRACT

The present invention relates to galenic formulations wherein the active ingredient aliskiren, preferably, a hemi-fumarate salt thereof, alone or in combination with another active ingredient, is melt-granulated and is present in an amount of more than 20% by weight based on the total weight of the oral dosage form, as well as a process of preparing said solid oral dosage form.

The present invention relates to solid oral dosage forms comprising an orally active renin inhibitor, aliskiren, or a pharmaceutically acceptable salt thereof, as the active ingredient. In particular, the present invention provides galenic formulations comprising aliskiren, preferably, a hemi-fumarate salt thereof, alone or in combination with another active agent. The present invention also relates to the processes for their preparation and to their use as medicaments.

In the following the term “aliskiren”, if not defined specifically, is to be understood both as the free base and as a salt thereof, especially a pharmaceutically acceptable salt thereof, most preferably a hemi-fumarate thereof.

Renin released from the kidneys cleaves angiotensinogen in the circulation to form the decapeptide angiotensin I. This is in turn cleaved by angiotensin converting enzyme in the lungs, kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume. Inhibitors of the enzymatic activity of renin bring about a reduction in the formation of angiotensin I. As a result a smaller amount of angiotensin II is produced. The reduced concentration of that active peptide hormone is the direct cause of, e.g., the antihypertensive effect of renin inhibitors. Accordingly, renin inhibitors, or salts thereof, may be employed, e.g., as antihypertensives or for treating congestive heart failure.

The renin inhibitor, Aliskiren, in particular, a hemi-fumarate thereof, is known to be effective in the treatment of reducing blood pressure irrespective of age, sex or race and is also well tolerated. Aliskiren in form of the free base is represented by the following formula

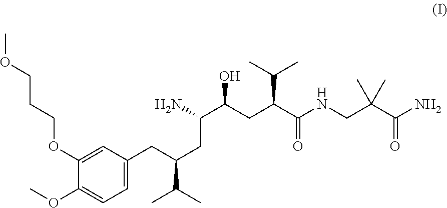

and chemically defined as 2(S),4(S),5(S),7(S)-N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide. As described above, most preferred is the hemi-fumarate salt thereof which is specifically disclosed in EP 678503 A as Example 83.

Administration of such a pharmaceutical agent via the oral route is preferred to parenteral administration because it allows self-administration by patients whereas parenteral formulations have to be administered in most cases by a physician or paramedical personnel.

However, Aliskiren is a drug substance difficult to formulate due to its physicochemical properties and it is not trivial to make oral formulations in the form of tablets in a reliable and robust way. In addition, in the particular case of high dose of Aliskiren or a pharmaceutically acceptable salt thereof (up to 300 mg of the free base per tablet) makes a high drug loading necessary in order to achieve a reasonable tablet size. Accordingly, there is a need for the development of suitable and robust galenical formulation overcoming these problems.

Examples of wet granulated Aliskiren formulations have been described and developed which employ a high drug loading of more than 46% by weight based on the total weight of the oral dosage form. Such a solid oral dosage form of Aliskiren is described e.g. in WO2005/089729. However, wet granulation methods are not attractive from an ecomomical standpoint since they require the use of granulation solvents and additional drying steps.

An alternative to the preparation of Aliskiren formulations by wet granulation techniques has been recently published in WO2008/116601. Such an alternative method for preparing aliskiren formulations is based on a process of hot-melt granulation. According to WO2008/116601, hot-melt granulation is a granulation process wherein the active ingredient and optional excipients are granulated with a binder which is in a molten state. According to the process in WO2008/116601, the granulating of aliskiren, or pharmaceutical salt thereof, alone or in admixture with at least one excipient, takes place with a binder which has a melting point lower than that of aliskiren, or salt thereof. As described on page 7 third paragraph of the specification, the melting point of said binder is at least 10° C. lower than the melting point of aliskiren, or salt thereof, and the temperature used in the granulation process may be in the rage between 40 to 90° C. (page 5, second paragraph). The invention is illustrated by the use of the binders Poloxamer 188 and 407, which have a melting point of 52-57° C., the binder PEG 4000, which has a melting point of 50-58° C., the binder Gelucire 50/13, which has a melting point of 40-48° C., and the binder Gelucire 44/14, which has a melting point of 44° C. Said binders are heated to a temperature 3° C. above their melting point (Table 1, step 2, page 18 in the specification), 5° C. above their melting point (Table 3, step 2, page 20 in the specification) or to 70° C. (Example 17, page 21 in the specification). In view of the above, the hot-melt granulation process disclosed in WO2008/116601 does not melt the active ingredient aliskiren, or salt thereof, instead it disperses aliskiren, or salt thereof, (in solid state) in the melted binder.

According to WO2008/116601, aliskiren changes to an amorphous state on its contact with water, which results in decreased stability of the product. Surprisingly, according to the present invention, it is found that robust galenical formulations of aliskiren, or of a pharmaceutically acceptable salt thereof, can be prepared by using a melt extrusion granulation process. As described herein below, melt extrusion granulation of aliskiren, or of a pharmaceutically acceptable salt thereof, relates to a granulation process wherein the extrudate temperature is at least the melting temperature of aliskiren, or salt thereof, and thus aliskiren, or salt thereof, melts. The melt extrusion of aliskiren, or salt thereof, and optionally one or more granulation excipients, according to the present invention, results in the conversion of aliskiren, or salt thereof, to its amorphous form and surprisingly results in an improvement of the tablet characteristics. Accordingly, the formulations according to the present invention are found to show less variability in the properties of the granules, such as density or particle size, and/or the properties of the tablet, such as friability, hardness, disintegration time or dissolution time, compared to the properties of the granules and/or tablet properties of formulations which involve, for example, a wet or a roller compaction granulation process. In particular, as regards the physical stability, the formulations of the present invention show no trend to re-crystallization or degradation upon a retest period of at least 6 months, when stored at or below 40° C./75% RH (room humidity) and 12 months when stored at 25° C./60% RH.

According to the present invention, it is also found that the drug loading that can be achieved when aliskiren is granulated by a melt extrusion granulation method is higher than that achievable by a wet granulation, a roller compaction method or a hot-melt granulation. In particular, in view of the examples described in WO2008/116601, the granulate obtainable by hot-melt granulation may contain of from 50% to 73% of aliskiren hemifumarate, and an aliskiren core tablet may contain of from 32% to 45% of aliskiren in the form of free base. As illustrated in the examples described herein, the granulate obtainable by melt granulation may contain of from 88% to 100% of aliskiren hemifumarate, and if the free base is used, the percentages will be adapted accordingly. Our process of melt extrusion granulation thus provides considerably higher loading of aliskiren in the granulate than that obtainable by hot-melt granulation according to WO2008/116601. As a consequence of such an increase in the loading of aliskiren in the granulate, the loading of aliskiren in the tablet is also higher than that obtainable by hot-melt granulation. As illustrated in the examples enclosed herein, an aliskiren core tablet prepared by melt extrusion granulation may contain, for example, of from 44% to 51% of aliskiren in the form of free base, and if a salts is used, such a the hemifumarate salt thereof, the percentages will be adapted accordingly. This loading is thus higher than that of the examples in WO2008/116601. Accordingly, the melt extrusion granulation provides, as a further benefit, means to reduce the tablet size, which can help improve patient compliance. In addition, the process of melt granulation is economically advantageous since it avoids the use of granulation solvents and provides means to continuous manufacturing.

The present invention provides galenic formulations wherein the active ingredient aliskiren, or a pharmaceutically acceptable salt thereof, preferably a hemi-fumarate salt thereof, is obtainable by melt-granulation, alone or in combination with one or more granulation excipient.

In one embodiment, the present invention relates to galenical formulations comprising as the only active ingredient aliskiren, or a pharmaceutical salt thereof, preferably a hemi-fumarate salt thereof, which is obtainable by melt-granulation, alone or in combination with one or more granulation excipient.

In another embodiment, the galenical formulations according to the present invention comprise an orally active renin inhibitor, Aliskiren, or a pharmaceutically acceptable salt thereof, (component (a)) and one further active ingredient (component (b)). In particular, the present invention relates to a pharmaceutical oral fixed dose combination comprising
a) a therapeutically effective amount of aliskiren, or a pharmaceutically acceptable salt thereof,
b) a therapeutically effective amount of valsartan, or a pharmaceutically acceptable salt thereof,

wherein the active ingredient aliskiren, or a pharmaceutically acceptable salt thereof, preferably a hemi-fumarate salt thereof, is obtainable by melt-granulation, alone or in combination with one or more granulation excipient.

In a solid oral dosage form according to the present invention, it is preferred if the active agent aliskiren, or a pharmaceutically acceptable salt thereof, is present in an amount ranging of from 75 mg to 600 mg of the free base per unit dosage form.

In a solid oral dosage form according to the present invention, it is preferred if the active agent aliskiren, or a pharmaceutically acceptable salt thereof, is present in an amount ranging of from 75 to 300 mg of the free base per unit dosage form.

In a solid oral dosage form according to the present invention, it is preferred if the active agent aliskiren, or a pharmaceutically acceptable salt thereof, is present in an amount ranging of from 150 to 300 mg of the free base per unit dosage form.

In a further preferred embodiment of the present invention, the dosage of aliskiren is in the form of a hemi-fumarate thereof and is present in an amount of about 83, about 166, about 332 or about 663 mg per unit dosage form, i.e. corresponding to 75 mg, 150 mg or 300 mg of the free base per unit dosage form.

In one embodiment, a galenical formulation according to the present invention comprises aliskiren as the only active agent in an amount of 20% or more, such as 40% or more, in particular 50% or more, for example of from 40% to 60% or of from 40% to 55%, based on the total weight of the oral dosage form. These percentages refer to the free base, and if a salts is used, such a the hemifumarate salt thereof, the percentages will be adapted accordingly.

In a further embodiment, a galenical formulation according to the present invention comprises aliskiren as the only active agent in an amount ranging of from 20 to 80%, such as of from 30 to 70%, such as of from 40 to 60%, in particular of from 40 to 55%, such as of from 44 to 52%, by weight based on the total weight of the oral dosage form. These percentages refer to the free base, and if a salt is used, such a the hemifumarate salt thereof, the percentages will be adapted accordingly.

In a preferred embodiment, a galenical formulation according to the present invention comprises aliskiren in an amount ranging of from 60 to 100%, such as of from 75 to 100%, such as of from 80 to 100%, for example of from 85% to 100%, by weight based on the total weight of the extrudate. These percentages refer to the free base, and if a salt is used, such a the hemifumarate salt thereof, the percentages will be adapted accordingly.

The general terms used hereinbefore and hereinafter preferably have within the context of this disclosure the following meanings, unless otherwise indicated:

For the purpose of this application, by the terms “melt extrusion” or “extrusion” or “melt extrusion granulation” or “melt granulation” it is meant an extrusion process where the extrudate temperature is at least the melting temperature, melting range, softening temperature or softening range of aliskiren hemifumarate, or of other aliskiren salt, or of aliskiren free base, or higher, such as 95° C. or higher, for example 100° C. or higher, such as 110° C. or higher, for example of from 95° C. to 140° C., in particular of from 100° C. to 120° C., such as of from 105° C. to 115° C. In the extrusion process, such a melt extrusion temperature results from the heating of the extruder screw or the heating of the extruder screw and the shear produced by the resistance between the material within the kneading screw; and at this temperature, aliskiren hemifumarate, or other salt of aliskiren, or aliskiren free base, melts or softens, thus resulting in the conversion of aliskiren, or salt thereof, to its amorphous form. In one embodiment, the extrusion process takes place in the presence of a granulation excipient, in particular a polymer, such as HPC, or mixture of polymers, that have a glass transition temperature, a glass transition range, a melting temperature or a melting range, in particular a glass transition temperature or glass transition range above the melting point, melting range, softening temperature or softening range of aliskiren hemifumarate, or other salt of aliskiren, in particular above the melting point, melting range, softening point or softening range of aliskiren hemifumarate, or of other aliskiren salt, or of aliskiren free base.

For the purpose of this application, the terms “melt extruded” or “extruded” or “melt granulated” mean conducting the process as defined in the preceding paragraph.

The terms “extrudate”, “melt extruded granulate” or “granulate obtainable by melt granulation” are used herein to mean the product obtained by the extrusion process.

The term “extrudate temperature” is used herein to mean the temperature of the material at the die exit as measured by a portable thermocouple plunged into one of the die openings.

The terms “melt extrusion temperature” or “extrusion temperature” are used herein to mean the temperature at which the extruder zone(s) is heated up.

The term “melt extrusion processing temperature” is used herein to mean the highest temperature at which a extruder zone is heated up.

As used herein, the term “glass transition temperature” or “softening temperature” means the temperature at which an amorphous or partially amorphous solid becomes soft on heating.

As used herein, the term “room temperature” or “ambient temperature” means a temperature of from 15 to 30° C., such as of from 20 to 30° C., such as of from 20 to 25° C.

As used herein the term “pharmaceutical composition” means a mixture containing a therapeutic compound to be administered to a mammal, e.g., a human in order to prevent, treat or control a particular disease or condition affecting the mammal. As used herein the term “pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

As used herein the term “therapeutic compound” means any compound, substance, drug, medicament, or active ingredient having a therapeutic or pharmacological effect, and which is suitable for administration to a mammal, e.g., a human, in a composition that is particularly suitable for oral administration. “Therapeutic compound”, “drug”, “active substance”, “active ingredient” “active agent” as used herein, refers to aliskiren unless specified otherwise.

The terms “effective amount” or “therapeutically effective amount” refers to the amount of the active ingredient or agent which halts or reduces the progress of the condition being treated or which otherwise completely or partly cures or acts palliatively on the condition.

In the above and in the following the term “Aliskiren”, if not defined specifically, is to be understood both as the free base and as a salt thereof, especially a pharmaceutically acceptable salt thereof, such as a hemi-fumarate salt thereof.

The term “disintegration” as used herein refers to a process where the pharmaceutical oral fixed dose combination, typically by means of a fluid, falls apart into separate particles and is dispersed. Disintegration is achieved when the solid oral dosage form is in a state in which any residue of the solid oral dosage form, except fragments of insoluble coating or capsule shell, if present, remaining on the screen of the test apparatus is a soft mass having no palpably firm core in accordance with USP<701>. The fluid for determining the disintegration property is water, such as tap water or deionized water. The disintegration time is measured by standard methods known to the person skilled in the art, see the harmonized procedure set forth in the pharmacopeias USP <701> and EP 2.9.1 and JP.

As used herein the term “granulation excipient” refers to any pharmaceutically acceptable material or substance that can be melt-extruded or melt-granulated with a therapeutic compound as further described below. The granulation excipient, for example, can be a polymer or a non-polymeric material. In one embodiment, the granulation excipient is a polymer.

As used herein the term “polymer” refers to a polymer or mixture of polymers that have a glass transition temperature, softening temperature or melting temperature by itself or in combination both above or below melting point (or melting range) of the therapeutic compound. The glass transition temperature (“Tg”) is the temperature at which such polymer's characteristics change from that of highly viscous to that of relatively less viscous mass. Types of polymers include, but are not limited to, water-soluble, water-swellable, water insoluble polymers and combinations of the foregoing.

Examples of polymers include, but are not limited to:
homopolymers and copolymers of N-vinyl lactams, e.g., homopolymers and copolymers of N-vinyl pyrrolidone (e.g., polyvinylpyrrolidone), copolymers of N-vinyl pyrrolidone and vinyl acetate or vinyl propionate;
   cellulose esters and cellulose ethers (e.g., methylcellulose and ethylcellulose) hydroxyalkylcelluloses (e.g., hydroxypropylcellulose), hydroxyalkylalkylcelluloses (e.g., hydroxypropylmethylcellulose), cellulose phthalates (e.g., cellulose acetate phthalate and hydroxylpropylmethylcellulose phthalate) and cellulose succinates (e.g., hydroxypropylmethylcellulose succinate or hydroxypropylmethylcellulose acetate succinate);
   high molecular polyalkylene oxides such as polyethylene oxide and polypropylene oxide and copolymers of ethylene oxide and propylene oxide (e.g. poly(propylene oxide) flanked by chains of poly(ethylene oxide), also known by the trade name pluronics);
   polyacrylates and polymethacrylates (e.g., methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates));
   polyacrylamides;
   vinyl acetate polymers such as copolymers of vinyl acetate and crotonic acid, partially hydrolyzed polyvinyl acetate;
   polyvinyl alcohol; and
   oligo- and polysaccharides such as carrageenans, galactomannans and xanthan gum, or mixtures of one or more thereof.

In one embodiment the polymer is selected from the group consisting of polyalkylene oxides, polyvinylpyrrolidone, such as PVPK 30, cellulose polymers, such as hydroxypropylmethylcellulose (e.g. HPMC 3cps) and hydroxypropyl cellulose (e.g. HPC-EXF) or mixtures thereof. Most preferably the polymer is hydroxypropyl cellulose (e.g. HPC-EXF). When present, the ratio of aliskiren to polymer is preferably of from 80:20 to 98:2, such as of from 85:15 to 96:4, such as 86:14, 90:10, 92:8, 95:5, in particular 92:8, based on the free base, and if a salt is used, the percentages will be adapted accordingly.

Non-polymeric granulation excipients include, but are not limited to, esters, hydrogenated oils, oils, natural waxes, synthetic waxes, hydrocarbons, fatty alcohols, fatty acids, monoglycerides, diglycerides, triglycerides and mixtures thereof. In one embodiment the non-polymeric granulation excipient is a fatty acid, for example stearic acid.

Examples of esters, such as glyceryl esters include, but are not limited to, glyceryl monostearate, e.g., CAPMUL GMS from Abitec Corp. (Columbus, Ohio); glyceryl palmitostearate; acetylated glycerol monostearate; sorbitan monostearate, e.g., ARLACEL 60 from Uniqema (New Castle, Del.); and cetyl palmitate, e.g., CUTINA CP from Cognis Corp. (Dusseldorf, Germany), magnesium stearate and calcium stearate.

Examples of hydrogenated oils include, but are not limited to, hydrogenated castor oil; hydrogenated cottonseed oil; hydrogenated soybean oil; and hydrogenated palm oil. An example of oil includes sesame oil.

Examples of waxes include, but are not limited to, carnauba wax, beeswax and spermaceti wax. Examples of hydrocarbons include, but are not limited to, microcrystalline wax and paraffin. Examples of fatty alcohols, i.e., higher molecular weight nonvolatile alcohols that have of from 14 to 31 carbon atoms include, but are not limited to, cetyl alcohol, e.g., CRODACOL C-70 from Croda Corp. (Edison, N.J.); stearyl alcohol, e.g., CRODACOL S-95 from Croda Corp; lauryl alcohol; and myristyl alcohol. Examples of fatty acids which may have of from 10 to 22 carbon atoms include, but are not limited to, stearic acid, e.g., HYSTRENE 5016 from Crompton Corp. (Middlebury, Conn.); decanoic acid; palmitic acid; lauric acid; and myristic acid.

Aliskiren, or a pharmaceutically acceptable salt thereof, can, e.g., be prepared in a manner known per se, especially as described in EP 678503 A, e.g., in Example 83.

A solid oral dosage form comprises a capsule or more preferably a tablet or a film-coated tablet.

A solid oral dosage form according to the invention comprises additives or excipients that are suitable for the preparation of the solid oral dosage form according to the present invention. Tabletting aids, commonly used in tablet formulation can be used and reference is made to the extensive literature on the subject, see in particular Fiedler's “Lexicon der Hilfstoffe”, 4th Edition, ECV Aulendorf 1996. These pharmaceutically acceptable additives include, but are not limited to, fillers or diluents, binders, disintegrants, lubricants, glidants, stabilising agents, surfactants, film-formers, softeners, pigments and the like. The amount of each additive in a pharmaceutical oral fixed dose combination may vary within ranges conventional in the art.

Suitable fillers include, without limitation, microcrystalline cellulose (e.g., cellulose MK), mannitol, sucrose or other sugars or sugar derivatives, calcium hydrogen phosphate qualities, starch qualities, preferably corn starch, low-substituted hydroxypropyl cellulose, hydroxyethyl cellulose, hydroxypropyl methyl cellulose, and combinations thereof, preferably, microcrystalline cellulose, e.g., products available under the registered trade marks AVICEL, FILTRAK, HEWETEN or PHARMACEL. When present, a filler may be employed in an amount ranging of from 10% to 50%, preferably of from 12% to 45%, more preferably of from 15% to 40% by weight of the dosage form (prior to any optional film coating).

Suitable binders include, without limitation, polyvinylpyrrolidone (PVP), such as e.g., PVP K 30 or PVP9OF, polyethylene glycols (PEG), e.g., PEG 4000, hydroxypropylmethyl cellulose, hydroxypropyl cellulose, pregelatinized starch and combinations thereof. A binder may be employed in an amount ranging of from 0.01% to 50%, preferably of from 0.01% to 10%, more preferably of from 0.1% to 5%, by weight of the dosage form (prior to any optional film coating).

Suitable lubricants include, without limitation, magnesium stearate, aluminum or calcium silicate, stearic acid, CUTINA (Hydrogenated Castor Oil), PEG 4000-8000, talc, glyceryl behenate, sodium stearyl fumarate (PRUV) and combinations thereof, preferably magnesium stearate. When present, a lubricant may be employed in an amount ranging of from 0.1% to 10%, preferably of from 0.5% to 5%, more preferably of from 1.1% to 3.3% by weight of the dosage form (prior to any optional film coating).

Suitable disintegrants include, without limitation, carboxymethylcellulose calcium (CMC-Ca), carboxymethylcellulose sodium (CMC-Na), crosslinked PVP (e.g. CROSPOVIDONE, POLYPLASDONE or KOLLIDON XL), alginic acid, sodium alginate and guar gum, most preferably crosslinked PVP (PVP XL, CROSPOVIDONE), crosslinked CMC (Ac-Di-Sol), carboxymethylstarch-Na (PIRIMOJEL and EXPLOTAB) or combinations thereof. Most preferred disintegrants are crosslinked PVP, preferably PVPP XL and/or carboxymethylstarch-Na. When present, the disintegrant(s) may be employed in an amount ranging of from 5% to 30%, preferably of from 10% to 25%, by weight of the dosage form (prior to any optional film coating).

Suitable glidants include, without limitation, colloidal silicon dioxide (e.g., Aerosil 200), magnesium trisilicate, powdered cellulose, talc and combinations thereof. Most preferred is colloidal silicon dioxide. When present and considering starch not as a glidant, a glidant may be employed in an amount ranging of from 0.05% to 5%, preferably of from 0.1% to 1%, by weight of the dosage form (prior to any optional film coating).

The solid oral dosage forms of the present invention have a low friability as is not more than 0.8%, preferably not more than 0.6%. The friability is measured by standard methods known to the person skilled in the art, see the harmonized procedure set forth in the pharmacopeias USP <1216>and EP 2.9.7 and JP.

The solid oral dosage forms of the present invention have also suitable hardness (e.g. an average hardness ranging of from 180 N to 310 N, such as of from 180 N to 250 N, in particular of from 200 N to 250 N). Such an average hardness is determined prior to the application of any film coating on the solid dosage form. Hardness may be measured according to a process described in The European Pharmacopoeia 4, 2.9.8 on page 201. The test employs apparatus consisting of 2 opposing jaws, one of which moves towards the other. The flat surfaces of the jaws are perpendicular to the direction of movement. The crushing surfaces of the jaws are flat and larger than the zone of contact with the tablet. The apparatus is calibrated using a system with a precision of one Newton. The tablet is placed between the jaws. For each measurement, the tablet is oriented in the same way with respect to the direction of the applied force. Measurements are carried out on 10 tablets. Results are expressed in terms of the mean, minimum and maximum values (in Newtons) of the force needed to crush the tablets.

A preferred embodiment of this invention is directed to the solid oral dosage forms which are film-coated. Suitable film coatings are known and commercially available or can be made according to known methods. Typically film coating materials are hydrophilic polymers such as polyethylene glycol, polyvinylpyrrolidone, polyvinyl alcohol, hydroxypropylcellulose, hydroxymethylcellulose, and hydroxypropylmethylcellulose or the like, of which hydroxypropyl methylcellulose is preferred. The film coating composition ingredients may include plasticizers, e.g., polyethylene glycols (e.g. polyethylene glycol 6000), triethylcitrate, diethyl phthalate, propylene glycol, glycerin in conventional amounts, as well as opacifiers such as titanium dioxide, and colorants, e.g. iron oxide, aluminum lakes, etc. Typically, a film coating material is applied in such an amount as to provide a film coating that ranges of from 1% to 6% by weight of the solid oral dosage form. Dry mixtures such as Sepifilm or Opadry mixtures prepared by Colorcon Corp. are preferably being used. These products are individually prepared dry pre-mixtures of film forming polymers, opacifiers, colorants and plasticizers which are further processed to aqueous film coating suspensions.

The film coating may be generally applied to achieve a weight increase of the solid oral dosage form of from 1 to 10 wt. %, and preferably of from 2 to 6 wt. %.

The film coating can be applied by conventional techniques in a suitable coating pan or fluidized bed apparatus using water and/or conventional organic solvents (e.g., methyl alcohol, ethyl alcohol, isopropyl alcohol), ketones (acetone), etc.

A further embodiment of the present invention is a process for the manufacture of a solid oral dosage form according to the present invention. Such a solid oral dosage form can be prepared by the following method, comprising the steps of: (1) melt extruding Aliskiren, or a pharmaceutical acceptable salt thereof, and optionally one or more granulation excipient, to form an aliskiren granulate; (2) optionally mixing the granulates with further pharmaceutically acceptable additives; (3) optionally compressing the final blend into a tablet; and (4) optionally, film coating the obtained tablet.

More particularly, the manufacturing process comprises the steps of
(a) blending Aliskiren, or a pharmaceutical acceptable salt thereof, and optionally one or more granulation excipient, to give a preblended material;
(b) sieving the blended material to give a screened material;
(c) blending the sieved material to give a blended material;
(d) melt extruding the blended material to give an extrudate;
(e) cooling the extrudate to ambient temperature, for example to a temperature of from 15 to 25° C., such as of from 20 to 25° C., such as 22 or 23° C., for example by using a chiller flaker unit, for example one available from BBA Cooler;
(f) milling the cooled extrudate;
(g) optionally blending the milled extrudate with one or more pharmaceutically acceptable excipients to form a final blended material;
(h) optionally compressing the final blend to form a tablet; and
(i) optionally applying a film coat in order to obtain the film coated tablets.

In one embodiment step (d) takes place according to a method comprising the following steps, preferably by using a 50 mm extruder:
(d1) optionally preheating of the extruder, preferably with preheating of the extruder, prior to feeding the material, preferably at a extrusion temperature such as; zones 1-3 of from 25° C. to 30° C., such as 25° C., zone-4 of from 50° C. to 80° C., such as 50° C., zone-5 of from 60° C. to 80° C., such as 60° C., zone-6 of from 70° C. to 100° C., such as 70° C., zones 7-8 of from 80° C. to 120° C., such as 80° C. and zones 9-10 of from 60° C. to 120° C., such as 60° C.,
(d2) running the extrusion process, preferably at a extrusion temperature such as; zones 1-3 of from 25° C. to 70° C., such as of from 25° C. to 35° C., such as 30° C., zones 4-6 of from 45° C. to 90° C., such as of from 45° C. to 55° C., such as 50° C., zones 7-8 of from 45° C. to 90° C., such as of from 45° C. to 55° C., such as 50° C. and zones 9-10 of from 40° C. to 120° C., such as of from 40° C. to 50° C. such as 45° C.

In another embodiment step (d) preferably takes place by using a 16 mm extruder, preferably without preheating of the extruder, preferably running the extrusion process at an extrusion temperature such as; zone-1 of from 25° C. to 55° C., such as of from 25° C. to 30° C., such as 25° C., zone-2 of from 25° C. to 70° C., such as of from 25° C. to 30° C., such as 25° C., zone-3 of from 25° C. to 90° C., such as of from 25° C. to 30° C., such as 25° C., zone-4 of from 30° C. to 130° C., such as of from 30° C. to 50° C., such as 40° C. and zone-5 of from 50° C. to 130° C., such as of from 50° C. to 80° C., such as 70° C.

In another embodiment step (d) preferably takes place by using a 27 mm extruder, preferably without preheating of the extruder, preferably running the extrusion process at an extrusion temperature such as; zones 1-3 of from 25° C. to 50° C., such as of from 25° C. to 35° C., such as 30° C., zone-4 of from 25° C. to 50° C., such as of from 25° C. to 40° C., such a 35° C., zone-5 of from 25° C. to 50° C., such as of from 25° C. to 40° C., such as 35° C., zone-6 of from 40° C. to 70° C., such as of from 40° C. to 50° C., such as 45° C. and zones 7-8 of from 40° C. to 70° C., such as of from 40° C. to 50° C., such as 45° C.

In a further embodiment, step (d) preferably takes place according to a method comprising the following steps, preferably by using a 50 mm extruder:
(d1) optionally preheating of the extruder, preferably with preheating of the extruder, prior to feeding the material, at a extrusion temperature such as; zones 1-3 25° C., zone 4 50° C., zone-5 60° C., zone-6 70° C., zones 7-8 80° C. and zones 9-10 60° C.,
(d2) running the extrusion process, preferably at a extrusion temperature such as; zones 1-3 30° C., zones 4-6 50° C., zones 7-8 50° C. and zones 9-10 45° C.

In a still further embodiment, step (d) preferably takes place by using a 16 mm extruder, preferably running the extrusion process at an extrusion temperature such as; zone-1 25° C., zone-2 25° C., zone-3 25° C. and zone-4 40° C. and zone-5 70° C.

In a still further embodiment, step (d) preferably takes place by using a 27 mm extruder, preferably running the extrusion process at an extrusion temperature such as; zones 1-3 30° C., zone-4 35° C., zone-5 35° C., zone-6 45° C. and zones 7-8 of 45° C.

In a preferred embodiment, the melt extrusion operation utilizes a 50 mm, a 27 mm or a 16 mm extruder, preferably wherein the material is fed at a rate of from 1 to 80 Kg/h, preferably of from 1 to 60 Kg/h, such as 1 Kg/h, 9 Kg/h or 50 Kg/h.

In a preferred embodiment, in step d) the temperature of the extrudate is of 95° C. or higher, for example 100° C. or higher, such as 110° C. or higher, for example of from 95° C. to 140° C., in particular of from 100° C. to 120° C., such as of from 105° C. to 115° C. Thus, preferably, in step c) the extrudate is cooled from such a preferred temperature, at which the extrudate is obtained in step d), to ambient temperature.

Once the extrudates are obtained, they may be formulated into oral forms, e.g., solid oral dosage forms, such as tablets, pills, lozenges, caplets, capsules or sachets, by adding additional conventional excipients which comprise an external phase of the pharmaceutical composition. The external phase of the pharmaceutical composition can also comprise an additional therapeutic compound. Such solid oral dosage forms, e.g., are unit oral dosage forms. Examples of such excipients include, but are not limited to, release retardants, plasticizers, disintegrants, binders, lubricants, glidants, stabilizers, fillers and diluents, in particular excipients described in the relevant chapters of The Handbook of Pharmaceutical Excipients, 4^{th} edition, Rowe et al., Eds., American Pharmaceuticals Association (2003); and Remington: the Science and Practice of Pharmacy, 21^{st} edition, Lippincott Williams & Wilkins (2005). One of ordinary skill in the art may select one or more of the aforementioned excipients with respect to the particular desired properties of the solid oral dosage form by routine experimentation and without any undue burden. The amount of each excipient used may vary within ranges conventional in the art.

In the manufacturing processes according to the present invention, attention is drawn to the numerous known methods of sieving, blending and mixing employed in the art, e.g., mixing in a free-fall or tumble blender, compressing into tablets on a single-punch or rotary tablet press or compaction on a roller compaction equipment. The sieving steps can be accomplished using any suitable means, e.g. using oscillating sieving or hand/vibrating sieves. The blending steps can be accomplished using any suitable means. Typically, Aliskiren or the Aliskiren granulate and pharmaceutically acceptable additives are dispatched to a suitable vessel such as a diffusion blender or diffusion mixer.

The milling/screening steps can be accomplished using any suitable means, such as milling through a screening mill or oscillating sieve/mill with a screen of at least 1.0 mesh size, such as 1.0 or 1.2 mm. Preferably the milled material is blended, often with pharmaceutically acceptable additives such as lubricants, fillers, disintegrants and glidants (the “outer phase”), in a diffusion blender.

Oral pharmaceutical products, e.g. tablets, are often manufactured in a batch processing manner. This means that the drug products are made according to a single manufacturing order during the same cycle of manufacture. Batch processing may result in lower output quality/quantity, lesser flexibility and higher labor costs when compared to other manufacturing techniques. In contrast, continuous manufacturing allows for the manufacturing of end products from raw materials in a single continuous fashion such as the output is maintained at a consistent rate.

In a preferred embodiment, the manufacture of solid oral dosage form pharmaceutical compositions according to the present invention relates to a melt extrusion continuous process. Said continuous process utilizes an equipment train that features various pieces of equipment for unit operations, such as mixing, sieving, granulating, milling, compressing, tableting or coating, linked together via transfer means, such as vacuum, gravity, convey belts, vibratory belts or bucket belts. The pharmaceutical materials (i.e., the raw materials, such as aliskiren or salt thereof, one or more pharmaceutically acceptable excipients or a mixture of the foregoing, intermediate drug products and final drug product) are continuously conveyed from one piece of unit operation equipment to the next piece of unit operation equipment without any intervention or assistance from a human operator of the equipment train. Therefore, the final result is a concatenation of a chain of independent unit operations into a single equipment train that allows for the feeding of raw materials into the equipment train upstream and having a solid oral dosage form, such as tablets, pills, caplets, capsules or sachets, preferably tablets, produced downstream.

An exemplary equipment train can comprise, for example, the following pieces: a blender; a extruder; a mill; and a tablet press. Any type of blender as known by one of ordinary skill may be used in the present invention, for example a bin blender. The extruder used in the present invention is configured for melt granulation. In general, a extruder includes a rotating screw(s) within a stationary barrel. Along the entire length of the screw, distributive kneading of the materials (e.g. aliskiren, or salt thereof, and optionally one or more granulation excipient) is provided by the rotation of the screw(s) within the barrel. The output of the extrude, extrudates, is transferred to a cooling tower. The cooling tower cools the extrudates to ambient temperature and once cooled, the extrudates may be transferred to an in-line mill for milling into granules. Preferably the extruder of the present invention is a twin-screw extruder, for example a 50 mm, 27 mm or a 16 mm twin-screw extruder. Any type of mill as known by one of ordinary skill may be used in the present invention, for example a Frewitt hammer mill using 1 mm or 2 mm screen with a rate of 2000 rpm. Any type of tablet press as known by one of ordinary skill in the art may also be used in the present invention. Examples of such tablet presses include, but are not limited to, low or high-speed presses, single/bi multilayer presses, and tablet-in-tablet presses. Tablet presses use forces between two and ninety kN to compress the milled materials.

In a preferred embodiment, the melt extrusion continuous process comprises, for example, the operations of extrusion, cooling, flaking and milling. Preferably the cooling operation utilizes a chiller flaker unit which cools the melted extrudate and cuts the formed solid sheets into small flakes. The flakes are conveyed into the mill, through a cooling tower, and are milled through a screen, for example a 2 mm screen.

In a further embodiment, the galenical formulations according to the present invention comprise an orally active renin inhibitor, Aliskiren, or a pharmaceutically acceptable salt thereof, and one or more further active ingredient.

In one embodiment, the galenical formulations according to the present invention comprise an orally active renin inhibitor (component (a)), Aliskiren, or a pharmaceutically acceptable salt thereof, and one further active ingredient (component (b)). In a preferred embodiment, component (b) is valsartan, or a salt thereof. In another preferred embodiment, component (b) is hydroclorothiazide (HCT) or is amlodipine, wherein “amlodipine” is to be understood both as the free base and as a salt thereof, especially a pharmaceutically acceptable salt thereof, most preferably a besylate salt thereof. Most preferably, amlodipine is used as the besylate salt thereof.

In another embodiment, the galenical formulations according to the present invention comprise an orally active renin inhibitor (component (a)), aliskiren, or a pharmaceutically acceptable salt thereof, and two further active ingredients (components (b) and (c)). In a preferred embodiment, component (b) is hydroclorothiazide (HCT) and component (c) is amlodipine, wherein “amlodipine” is as defined above.

Typically, the pharmaceutical oral fixed dose combination comprising components (a) and (b) is a solid dosage form, such as a mono-layer or a multilayer, such as a bi-layer, tablet.

In one embodiment according to the present invention, the components (a) and (b) are formulated together in the form of a monolayer. Monolayers according to the present invention can be manufactured, for example by a method comprising the steps of (1) granulating component (a) and pharmaceutically acceptable additives, as described above; (2) granulating component (b) and pharmaceutically acceptable additives, for example as described below, such as by roller compaction; (3) sieving respective granulates; (4) optionally mixing the respective granulates with outer phase excipients; (5) mixing respective granulates; (6) screening the material from step (5); (7) optionally, blending the obtained sieved material from (6) together with further pharmaceutically acceptable additives; (8) compressing the blend from (7) to form a monolayer tablet and (9) optionally, film coating the obtained monolayer tablet.

Pharmaceutically acceptable additives suitable for use in monolayer tablets, according to the present invention include, without limitation, diluents or fillers, disintegrants, glidants, lubricants, binders, colorants and combinations thereof, as defined above.

In one preferred embodiment, component (b) is granulated with pharmaceutically acceptable additives, optionally in the presence of a granulation liquid. The granulation liquid can be any liquid or liquid mixture well-known in the granulation art such as ethanol, a mixture of ethanol and water, a mixture of ethanol, water and isopropanol. The process is then referred to as an organic wet granulation. Wet granulation is typically accomplished by using the following method (1) blending component (b) and pharmaceutically acceptable additives in the presence of a granulation liquid to form a blended material; (2) drying the blended material, (3) sieving the blended material; and (4) screening the sieved material to isolate the wet-granulated fraction.

Alternatively, granulation of component (b) is accomplished by using a dry granulation method, which may comprise the following steps : (1) blending component (b) and pharmaceutically acceptable additives to form a blended material; (2) sieving the blended material; (3) blending the sieved material to form a final blend material; (4) compacting the final blend material to form a compacted material; (5) milling the compacted material to form a milled material; and (6) blending the milled material to form the dry-granulated fraction. Particularly preferable is a roller compaction method whereby the step of compacting is performed using a roller compactor. In this case, the compacting step can be accomplished using any suitable means. Typically, compacting is accomplished using a roller compactor with a compaction force (for development scale machines) ranging of from 2 kN to 6 kN i.O., preferably of from 3 to 5 kN. Compaction may also be carried out by slugging the blended powders into large tablets that are then size-reduced. Preferably, the device used is a Freund Corporation; Roller Compactor Type TF Mini. Using this equipment, the screw speed is suitably adjusted to ensure proper quality of the roller compacted material. Preferably, the screw speed is more than 15 rpm, such as 20 to 30 rpm. Moreover, using this equipment, the roll speed is suitably adjusted to ensure proper quality of the roller compacted material. Preferably, the roll speed is 3 to 5 rpm. It is also preferred that no pre-compression force is applied.

In another preferred embodiment component (b) is granulated by a melt extrusion granulation method. Melt extrusion granulation is typically accomplished by using the following method: (1) blending component (b) and optionally one or more granulation excipient to form a blended material; (2) sieving the blended material, (3) melt extruding the sieved material, (4) cooling the extrudate to ambient temperature, (5) milling the melt granulation material, (6) optionally blending the milled melt granulation material with sieved further pharmaceutically acceptable additives to give the final melt granulate.

In a preferred embodiment, component (b) is granulated by roller compaction.

In another embodiment according to the present invention, the components (a) and (b) are formulated in such a way that they are physically separated, for example they are formulated into separate layers e.g. a multi- or bilayer tablet, preferably a bilayer tablet. A multilayer tablet has at least two layers (bilayer tablet) or can have three, four, five or more layers. Each of the layers contains not more than one of the components. Preferably, the tablet has 2 layers with one of the components in one of the two layers, but it is also possible that in addition to these two layers the tablet contains further layers containing only carrier and which may function e.g. as separation layer(s) or outer coating layer(s). Alternatively, if more than two layers are present, the components may be present in more than one layer as long as they are not present together in the same layer. For practical purposes, a bilayer tablet is preferred but all information detailed below is equally applicable to multilayer tablets.

Multilayer tablets, in particular, bilayer tablets, according to the present invention are characterized in that one layer contains component (a) and the other layer contains component (b). According to the present invention, the layer containing component (a) is prepared by melt extrusion, as described hereinbefore. The layer containing component (b) may be prepared, for example, by granulation methods as described above.

Pharmaceutically acceptable additives suitable for use in multilayer tablets, in particular bilayer tablets, according to the present invention include, without limitation, diluents or fillers, disintegrants, glidants, lubricants, binders, colorants and combinations thereof, as defined above.

The multilayer, preferably bilayer, tablet pharmaceutical oral fixed dose combinations, according to the present invention, have low friability and suitable hardness (e.g. an average hardness ranging of from 180 N to 310 N, such as of from 250 N to 300 N or of from 200 N to 250 N; for bilayer forms). Preferably the friability is not more than 0.8%. The friability is measured by standard methods known to the person skilled in the art, see the harmonized procedure set forth in the pharmacopeias USP <1216> and EP 2.9.7 and JP. The average hardness is determined prior to the application of any film coating on the pharmaceutical oral fixed dose combinations.

In one embodiment, in a multilayer tablet, according to the present invention, such as a bilayer tablet, component (a) is present in an amount of 20% of more, such as 22% or more, such as 25% or more by weight based on the total weight of the pharmaceutical oral fixed dose combination. These percentages are based on the free base of component (a) and if a salt is used the percentages will be adapted accordingly.

In another embodiment, in a multilayer tablet, according to the present invention, such as a bilayer tablet, component (a) is present in an amount of 40% of more, such as 50% or more, such as 60% or more, by weight based on the total weight of the layer comprising component (a). These percentages are based on the free base of component (a) and if a salt is used the percentages will be adapted accordingly.

In a further embodiment, in a multilayer tablet, according to the present invention, such as a bilayer tablet, component (a) is present in an amount of from 40 to 70%, such as 50 to 65%, such as 50 to 60%, in particular of from 60 to 70%, by weight based on the total weight of the layer comprising component (a). These percentages are based on the free base of component (a) and if a salt is used the percentages will be adapted accordingly.

In a further embodiment, in a multilayer tablet, according to the present invention, such as a bilayer tablet, component (a) is present in an amount of from 70 to 100%, such as 75 to 98%, such as 85 to 98%, by weight based on the total weight of the extrudate comprising component (a). These percentages are based on the free base of component (a) and if a salt is used the percentages will be adapted accordingly.

In a still further embodiment, in a multilayer tablet, according to the present invention, such as a bilayer tablet, component (b) is present in an amount of 20% of more, such as 23% or more, such as 25% or more, such as 28% or more, by weight based on the total weight of the pharmaceutical oral fixed dose combination. These percentages are based on the free acid or free base of component (b) and if a salt is used the percentages will be adapted accordingly.

In yet another embodiment, in a multilayer tablet, according to the present invention, such as a bilayer tablet, component (b) is present in an amount of 50% of more, by weight based on the total weight of the layer comprising component (b). These percentages are based on the free acid or free base of component (b) and if a salt is used the percentages will be adapted accordingly.

In a still further embodiment, in a multilayer tablet, according to the present invention, such as a bilayer tablet, component (b) is present in an amount of from 30% to 70% by weight based on the total weight of the layer comprising component (b). These percentages are based on the free acid or free base of component (b) and if a salt is used the percentages will be adapted accordingly.

In a preferred embodiment component (b) is valsartan, thus the percentages of component (b) refer to the free acid, and if a salt thereof is used the percentages will be adapted accordingly.

The details regarding the components (a) and (b) and pharmaceutically acceptable additives, i.e., source, amount, etc., are as set forth hereinabove and hereinafter.

The multilayer, preferably a bilayer, tablet according to the present invention can be prepared, for example, by a method comprising the steps of (1) melt granulating component (a) as described above, to form an Aliskiren granulate; (2) granulating component (b), for example as described herein; (3) sieving the respective granulates; (4) optionally mixing the respective granulates with outer phase excipients; and (5) compressing the granulates, of both components (a) and (b), together to form a bilayer tablet. Typically compression is accomplished using a bilayer rotary tablet press. Typical compression force ranges of from 12 kN to 45 kN. Preferably, the layer containing component (b) is pre-compressed and the layer containing component (a) is added to the resulting pre-compressed layer and then both layers are compressed. Optionally, the method comprises the step of film coating the multilayer, preferably bilayer, tablet. Film coating can be accomplished using any suitable means. Suitable film coatings are known and commercially available or can be made according to known methods. Typically the film coating material is a polymeric film coating material comprising materials such as hydroxypropylmethyl cellulose, polyethylene glycol, talc and colorant. Typically, a film coating material is applied in such an amount as to provide a film coating that ranges of from 1% to 6% by weight of the film-coated tablet.

The blending, drying, sieving and mixing steps can be accomplished by using any suitable means known in the art. Typically blending and mixing steps are accomplished by using diffusion blenders or diffusion mixers, respectively. The sieving steps can be accomplished, for example, by using oscillating sieving.

The term “Valsartan”, if not defined specifically, is to be understood both as the free base and as a salt thereof, especially a pharmaceutically acceptable salt thereof. Valsartan , or a pharmaceutically acceptable salt thereof, can, e.g., be prepared in a manner known per se, for example as described in WO2004/026847, in WO2005/014602 and in U.S. Pat. No. 5,399,578.

Preferred salts forms include acid addition salts. The compounds having at least one acid group (e.g., COOH or 5-tetrazolyl) can also form salts with bases. Suitable salts with bases are, e.g., metal salts, such as alkali metal or alkaline earth metal salts, e.g., sodium, potassium, calcium or magnesium salts, or salts with ammonia or an organic amine, such as morpholine, thiomorpholine, piperidine, pyrrolidine, a mono-, di- or tri-lower alkylamine, e.g., ethyl-, tert-butyl-, diethyl-, diisopropyl-, triethyl-, tributyl- or dimethyipropylamine, or a mono-, di- or trihydroxy lower alkylamine, e.g., mono-, di- or tri-ethanolamine. Corresponding internal salts may furthermore be formed. Salts which are unsuitable for pharmaceutical uses but which can be employed, e.g., for the isolation or purification of free compounds I or their pharmaceutically acceptable salts, are also included. Even more preferred salts are, e.g., selected from the mono-sodium salt in amorphous form; di-sodium salt of Valsartan in amorphous or crystalline form, especially in hydrate form, thereof. Mono-potassium salt of Valsartan in amorphous form; di-potassium salt of Valsartan in amorphous or crystalline form, especially in hydrate form, thereof.

Calcium salt of Valsartan in crystalline form, especially in hydrate form, primarily the tetrahydrate thereof; magnesium salt of Valsartan in crystalline form, especially in hydrate form, primarily the hexahydrate thereof; calcium/magnesium mixed salt of Valsartan in crystalline form, especially in hydrate form; bis-diethylammonium salt of Valsartan in crystalline form, especially in hydrate form; bis-dipropylammonium salt of Valsartan in crystalline form, especially in hydrate form; bis-dibutylammonium salt of Valsartan in crystalline form, especially in hydrate form, primarily the hemihydrate thereof; mono-L-arginine salt of Valsartan in amorphous form; bis-L-arginine salt of Valsartan in amorphous form; mono-L-lysine salt of Valsartan in amorphous form; bis-L-lysine salt of Valsartan in amorphous form. Most preferably, Valsartan is used as the free acid. Valsartan granulation can be accomplished by any suitable means. In a preferred embodiment of this invention, Valsartan granulation is accomplished by (1) blending component (b) and pharmaceutically acceptable additives to form a blended material; (2) sieving the blended material; (3) blending the sieved material to form a final blend material; (4) compacting the final blend material to form a compacted material; (5) milling the compacted material to get a milled material; and (6) blending the milled material to form the Valsartan granulate.

The blending of step (1 and 3) can be accomplished using any suitable means. Typically the component b) and pharmaceutically acceptable additives are dispatched to a suitable vessel such as a diffusion blender or diffusion mixer. The sieving of step (2) can be accomplished using any suitable means such as those described above. The compaction of step (4) can be accomplished using any suitable means. For example, typically for component b) compacting is accomplished using a roller compactor with a compaction force ranging of from 20 kN to 60 kN, preferably 35 kN. Compaction may also be carried out by slugging the blended powders into large tablets that are then size-reduced. The milling of step (5) can be accomplished using any suitable means. Typically the compacted material is milled through a screening mill. The blending of step (6) can be accomplished using any suitable means. Preferably the milled material is blended, often with a pharmaceutically acceptable additive such as a lubricant, in a diffusion blender.

It is preferred that valsartan is present in an amount ranging of from 75 to 350mg, such as of from 100 to 200 mg, more preferably of from 80 mg to 320 mg, such as of from 160 to 320 mg, per unit dosage form, in particular 80, 160 or 320 mg, such as 160 or 320 mg, based on the free acid, and if a salt is used, the percentages will be adapted accordingly.

In a preferred embodiment of the present invention, valsartan is present in an amount of from 15 to 40%, such as of from 20 to 40%, such as of from 20 to 30%, by weight based on the total weight of the pharmaceutical oral fixed dose combination. These percentages are based on the free acid and if a salt is used, the percentages will be adapted accordingly.

The weight ratio of aliskiren to valsartan preferably ranges of from 1:0.001 to 1:5, more preferably of from 1:0.5 to 1:4 or 1:0.03 to 1:0.07. Most preferably, the weight ratio is of from 1:1.0 to1.1; 1:2.1 to 2.2; or 1:0.005 to 0.006 based on the free base of (a) and free acid of (b). Most preferably, components (a) and (b), are used in amounts of 75/80 mg, 75/160 mg, 150/80 mg, 150/160 mg, 300/320 mg, 300/160 mg or 150/320 mg, most preferably 150/160 mg, 300/320 mg, 300/160 mg or 150/320 mg of (a)/(b), based on the free base of (a) and free acid of (b). In one embodiment it is preferred to use a high drug load using 300 mg of (a) and/or 320 mg of (b), most preferably 300/320 mg of (a)/(b). When using a salt such as the hemifumarate for component (a), the ratios will be adapted accordingly.

The resulting formulations in accordance with the present invention show the following advantages:
A relatively high drug loading may easily be achieved;
The formulation of pharmaceutical oral fixed dose combinations with sufficient hardness, resistance to friability, disintegration time etc. is possible;
The sticking tendency and poor flow of the drug substance is reduced to a minimum;
A robust manufacturing process is achieved;
Scale-up of formulation and process resulting in a reproducible performance is achieved;
Sufficient stability to achieve a reasonable shelf life is achieved;
Shorter processing times are achieved in particular because the process of melt granulation circumvents the use of granulation solvents and thus avoids any drying steps therefore rendering the process more economic; and
Formulations wherein aliskiren is in its amorphous form are achieved.

The invention likewise relates to a process for the preparation of solid oral dosage forms as described herein above. Such a solid oral dosage form may be produced by working up components as defined herein above in the appropriate amounts, to form unit solid oral dosage forms.

The solid oral dosage forms of the present invention are useful for lowering the blood pressure, either systolic or diastolic or both. The conditions for which the instant invention is useful include, without limitation, hypertension (whether of the malignant, essential, reno-vascular, diabetic, isolated systolic, or other secondary type), congestive heart failure, angina (whether stable or unstable), myocardial infarction, artherosclerosis, diabetic nephropathy, diabetic cardiac myopathy, renal insufficiency, peripheral vascular disease, left ventricular hypertrophy, cognitive dysfunction (such as Alzheimer's) and stroke, headache and chronic heart failure.

The present invention likewise relates to a method of treating hypertension (whether of the malignant, essential, reno-vascular, diabetic, isolated systolic, or other secondary type), congestive heart failure, angina (whether stable or unstable), myocardial infarction, artherosclerosis, diabetic nephropathy, diabetic cardiac myopathy, renal insufficiency, peripheral vascular disease, left ventricular hypertrophy, cognitive dysfunction, e.g., Alzheimer's, stroke, headache and chronic heart failure comprising administering to an animal, including human patient, in need of such treatment a therapeutically effective solid oral dosage form according to the present invention.

The present invention likewise relates to the use of a solid oral dosage form according to the present invention for the manufacture of a medicament for the treatment of hypertension (whether of the malignant, essential, reno-vascular, diabetic, isolated systolic, or other secondary type), congestive heart failure, angina (whether stable or unstable), myocardial infarction, artherosclerosis, diabetic nephropathy, diabetic cardiac myopathy, renal insufficiency, peripheral vascular disease, left ventricular hypertrophy, cognitive dysfunction, e.g., Alzheimer's, stroke, headache and chronic heart failure.

The present invention likewise relates to a pharmaceutical composition for the treatment of hypertension (whether of the malignant, essential, reno-vascular, diabetic, isolated systolic, or other secondary type), congestive heart failure, angina (whether stable or unstable), myocardial infarction, artherosclerosis, diabetic nephropathy, diabetic cardiac myopathy, renal insufficiency, peripheral vascular disease, left ventricular hypertrophy, cognitive dysfunction, e.g., Alzheimer's, stroke, headache and chronic heart failure, comprising a solid oral dosage form according to the present invention.

Ultimately, the exact dose of the active agent and the particular formulation to be administered depend on a number of factors, e.g., the condition to be treated, the desired duration of the treatment and the rate of release of the active agent. For example, the amount of the active agent required and the release rate thereof may be determined on the basis of known in vitro or in vivo techniques, determining how long a particular active agent concentration in the blood plasma remains at an acceptable level for a therapeutic effect.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way.

### EXAMPLES

The following manufacturing process is meant to show a method of practicing the present invention. The extruders below mentioned serve to illustrate the invention without limiting the scope thereof, while they on the other hand represent preferred embodiments for effecting the melt extruding step d). Likewise, the temperature of the extruder zones are illustrative.

Aliskiren, or a pharmaceutical acceptable salt thereof, and optionally one or more granulation excipient, is blended in a bin blender for two hundred rotations to give a preblended material. The blend is sieved through a 2 mm screen to give a screened material and next is blended for two hundred rotations more to give a blended material. The blend is introduced into the feed section, or hopper, of a twin screw extruder. A suitable twin screw extruder is, for example, the 50 mm, the 27 mm or the 16 mm Leistritz extruder. Before feeding the material, the extruder may be preheated as indicated below. Upon preheating, if preheating is required, the extruder is heated to the melt extrusion temperatures illustrated below. The screw speed is set and the volumetric feed rate is adjusted. The heating of the extruder screw and the shear produced by the resistance between the material within the kneading screw allows for the melting of aliskiren hemifumarate, or of other aliskiren salt. The extrudates, or granules, are cooled to ambient temperature by using a chiller flaker unit, for example one available from BBA Cooler. The cool granules are then milled. A suitable miller is the Frewitt hammer mill using a 2 mm or 1 mm screen preferably set at 2000 rpm. For the external phase, the external phase excipients are first passed through a suitable mesh. For example, aerosil 200 is screened through a 1.0 mm screen, magnesium stearate is screened through a 0.9 mm screen, and indigotine lake is screened through a 0.5 mm screen. The excipients are then blended with the obtained granules using a suitable bin blender. The resulting final blend is compressed into tablets by using a conventional rotary tablet press at the appropriate compression force. 50 mm extruder: extruder preheating (for 15 min)

| | |
|---|---|
| Temp. zones 1-3 | 25° C. |
| Temp. zone 4 | 50° C. |
| Temp. zone 5 | 60° C. |
| Temp. zone 6 | 70° C. |
| Temp. zones 7-8 | 80° C. |
| Temp. zones 9-10 | 60° C. |

50 mm extruder: melt extrusion temperature conditions

| | | | | |
|---|---|---|---|---|
| Temp. zones 1-3 | 30° C. (±5° C.) | 25° C. | 30° C. | 35° C. |
| Temp. zones 4-6 | 50° C. (±5° C.) | 45° C. | 50° C. | 55° C. |
| Temp. zones 7-8 | 50° C. (±5° C.) | 45° C. | 50° C. | 55° C. |
| Temp. zones 9-10 | 45° C. (±5° C.) | 40° C. | 45° C. | 50° C. |
| Feed rate | 50 kg/h - fixed | ** | ** | ** |
| Screw speed | 150 rpm (±5 rpm) | 145 rpm | 150 rpm | 155 rpm |

In one embodiment (**) = 50 kg/h

27 mm extruder: melt extrusion temperature conditions (no preheating)

| | | | | |
|---|---|---|---|---|
| Temp. zones 1-3 | 30° C. (±5° C.) | 25° C. | 30° C. | 35° C. |
| Temp. zone 4 | 35° C. (±5° C.) | 45° C. | 50° C. | 55° C. |
| Temp. zone 5 | 35° C. (±5° C.) | 45° C. | 50° C. | 55° C. |
| Temp. zone 6 | 45° C. (±5° C.) | 40° C. | 45° C. | 50° C. |
| Temp. zones 7-8 | 45° C. (±5° C.) | 40° C. | 45° C. | 50° C. |
| Feed rate | 9 kg/h - fixed | *** | *** | *** |
| Screw speed | 150 rpm (±5 rpm) | 145 rpm | 150 rpm | 155 rpm |

In one embodiment (***) = 9 kg/h

16 mm extruder: melt extrusion temperature conditions (no preheating)

| | | | | |
|---|---|---|---|---|
| Temp. zone 1 | 25° C. | * | * | * |
| Temp. zone 2 | 25° C. | * | * | * |
| Temp. zone 3 | 25° C. | * | * | * |
| Temp. zone 4 | 40° C. (±5° C.) | 35° C. | 40° C. | 45° C. |
| Temp. zone 5 | 70° C. (±5° C.) | 65° C. | 70° C. | 75° C. |
| Feed rate | 1 kg/h - fixed | # | # | # |
| Screw speed | 140 rpm (±10 rpm) | 130 rpm | 140 rpm | 150 rpm |

In one embodiment (*) = 25° C. and (#) = 1 kg/h

| Milling/screening | Aliskiren | Cool | External phase |
|---|---|---|---|
| parameters | and polymer | Extrudate | excipients |
| screen (mm) | 2.0 | 2.0 | 0.5-1.0 |

### Example 1

### Bilayer Formulations With a Melt Granulated Aliskiren Layer

The Aliskiren layer is prepared as described above.

The components of the Valsartan layer are mixed, granulated and compressed as described herein. The Valsartan layer is filled into an eccentric tablet press for all bilayer variants and compressed with a compression force of <2.5kN. The Aliskiren layer is added on top of the Valsartan layer and then the tablet core is compressed with a compaction force of from 5 to 40kN to obtain a bilayer tablet core.

VARIANT 1. Melt extrudate: Aliskiren and HPC;

16 mm extruder, extrudate temperature 105-110° C.

| Aliskiren/ | | Composition | Composition |
|---|---|---|---|
| Valsartan | | per unit | per unit |
| 300/320 mg | | [mg/unit] | [%] |
| Aliskiren | Aliskiren hemifumarate | 331.50 | 29.59 |
| layer | HPC* | 45.20 | 4.03 |
| | Avicel 102 (MCC) | 68.05 | 6.07 |
| | Crospovidone XL | 50.00 | 4.46 |
| | Aerosil 200 | 2.50 | 0.22 |
| | Indigotin-farBlack | 0.50 | 0.04 |
| | Magnesium stearate | 2.50 | 0.22 |
| Valsartan | Valsartan | 320.00 | 28.57 |
| layer | Avicel 102 (MCC) | 229.50 | 20.49 |
| | Crospovidone XL | 46.50 | 4.15 |
| | Aerosil 200 | 6.00 | 0.54 |
| | Mg stearate (internal) | 12.00 | 1.07 |
| | Mg-Stearate (external) | 6.00 | 0.54 |
| | Total | 1120.25 | 100.00 |

| | |
|---|---|
| Mean Hardness [N] | 260 N (220-300 N) |
| Friability 10St./6.5 g 500 U [%] | 0.2% |
| Disintegration time for Valsartan | 6 min-8 min |
| layer [minutes] | |
| Disintegration time for Aliskiren | 22-29 min |
| layer [minutes] | |
| Dissolution profile of Aliskiren | Dissolution profile of Aliskiren |
| at pH 4.5 after 10 min | at pH 4.5 after 20 min |
| 22 | 46 |

HPC*hydroxypropylcellulose with aqueous viscosity of 300-600 mPas at 10% w/w concentration and 80,000 average molecular weight

VARIANT 2. Melt extrudate: Aliskiren and HPC;

16 mm extruder, extrudate temperature 105-110° C. 27 mm extruder, extrudate temperature 105-110° C. 50 mm extruder, extrudate temperature 105-115° C.

| Aliskiren/ | | Composition | Composition |
|---|---|---|---|
| Valsartan | | per unit | per unit |
| 150/160 mg | | [mg/unit] | [%] |
| Aliskiren | Aliskiren hemifumarate | 165.75 | 29.60 |
| layer | HPC* | 12.00 | 2.14 |
| | Cellulose MKGR | 44.63 | 7.97 |
| | Crospovidone | 25.00 | 4.46 |
| | Aerosil 200 | 1.25 | 0.22 |
| | Indigotin Lake 12196 | 0.13 | 0.02 |
| | Magnesium stearate | 1.25 | 0.22 |
| Valsartan | Valsartan | 160.00 | 28.57 |
| layer | Cellulose MK GR | 108.00 | 19.29 |
| | Crospovidone | 30.00 | 5.36 |
| | Aerosil 200 | 3.00 | 0.54 |
| | Mg stearate (internal) | 6.00 | 1.07 |
| | Mg stearate (external) | 3.00 | 0.54 |
| | Total | 560.0 | 100.00 |

| | |
|---|---|
| Mean Hardness [N] | 220 N (190-250) |
| Friability 10St./6.5 g 500 U [%] | 0.1% |
| Disintegration time for Valsartan | 3 min-6 min |
| layer [minutes] | |
| Disintegration time for Aliskiren | 15 min-19 min |
| layer [minutes] | |
| Dissolution profile of Aliskiren | Dissolution profile of Aliskiren |
| at pH 4.5 after 10 min | at pH 4.5 after 20 min |
| 31 | 65 |

HPC*hydroxypropylcellulose with aqueous viscosity of 300-600 mPas at 10% w/w concentration and 80,000 average molecular weight

VARIANT 3. Melt extrudate: Aliskiren and HPC; 16 mm extruder, extrudate temperature 105-110° C. 27 mm extruder, extrudate temperature 105-110° C. 50 mm extruder, extrudate temperature 105-115° C.

| Aliskiren/ | | Composition | Composition |
|---|---|---|---|
| Valsartan | | per unit | per unit |
| 300/320 mg | | [mg/unit] | [%] |
| Aliskiren | Aliskiren hemifumarate | 331.50 | 29.60 |
| layer | HPC* | 24.00 | 2.14 |
| | Cellulose MKGR | 89.25 | 7.97 |
| | Crospovidone | 50.00 | 4.46 |
| | Aerosil 200 | 2.50 | 0.22 |
| | Indigotin Lake 12196 | 0.25 | 0.02 |
| | Magnesium stearate | 2.50 | 0.22 |
| Valsartan | Valsartan | 320.00 | 28.57 |
| layer | Cellulose MK GR | 216.00 | 19.29 |
| | Crospovidone | 60.00 | 5.36 |
| | Aerosil 200 | 6.00 | 0.54 |
| | Mg stearate (internal) | 12.00 | 1.07 |
| | Mg stearate (external) | 6.00 | 0.54 |
| | Total | 1120.00 | 100.00 |

| | |
|---|---|
| Mean Hardness [N] | 240 (210-280 N) |
| Friability 10St./6.5 g 500 U [%] | 0.2% |
| Disintegration time for Valsartan | 3-6 min |
| layer [minutes] | |
| Disintegration time for Aliskiren | 17-22 min |
| layer [minutes] | |
| Dissolution profile of Aliskiren | Dissolution profile of Aliskiren |
| at pH 4.5 after 10 min | at pH 4.5 after 20 min |
| 27 | 59 |

HPC*hydroxypropylcellulose with aqueous viscosity of 300-600 mPas at 10% w/w concentration and 80,000 average molecular weight

### Example 2

### Melt Extrudate: Aliskiren Hemifumarate and PVP K30

Variant 1: 16 mm extruder, extrudate temperature 110-120° C.

| VARIANT 1 | mg/unit |
|---|---|
| Internal phase (Melt Extrudate) | |
| Aliskiren hemifumarate | 331.5 |
| PVP K30* | 14.7 |

Melt Extrusion Processing Temp. = 110° C.-LowShear-16 mm extruder

| | |
|---|---|
| External phase | |
| Polyvinylpyrrolidone XL | 96.4 |
| Microcrystalline cellulose Grn | 223.8 |
| Aerosil 200 | 3.6 |
| Magnesium stearate | 10.0 |
| Total Core tablet weight | 680 mg |
| Mean Hardness [N] | 213-228 |
| Friability [500 drops] | 0.07% |
| Disintegration time of core tablets | 15 min |
| Disintegration time of coated tablets | 17 min |

Dissolution profile of core tablets (% of drug release, n = 3 tablets)

| 15 min | 30 min | 60 min |
|---|---|---|
| 77.53 | 102.24 | 103.27 |

Dissolution profile of coated tablets (% of drug release, n = 3 tablets)

| 15 min | 30 min | 60 min |
|---|---|---|
| 69.15 | 102.67 | 103.48 |

Dissolution profile parameters
Dissolution media: 0.01N HCl
Volume of test media: 500 ml
Method: Basket method (100 rpm)
PVP K30*polyvinylpyrrolidone with aqueous viscosity of 5.5-8.5 mPas at 10% w/w concentration and 44,000-54,000 average molecular weight

Variant 2: 16 mm extruder, extrudate temperature 110-120° C.

| VARIANT 2 | mg/unit |
|---|---|
| Internal phase (Melt Extrudate) | |
| Aliskiren hemifumarate | 331.5 |
| PVP K30* | 24 |

Melt Extrusion Processing Temp. = 110° C.-Low Shear-16 mm extruder

| | |
|---|---|
| External phase | |
| Polyvinylpyrrolidone XL | 96.4 |
| Microcrystalline cellulose Grn | 214.5 |
| Aerosil 200 | 3.6 |
| Magnesium stearate | 10.0 |
| Total Core tablet weight | 680 mg |
| Mean Hardness [N] | 198-215 |
| Friability [500 drops] | 0.2% |
| Disintegration time of core tablets | 16 min |
| Disintegration time of coated tablets | 18-19 min |

Dissolution profile of core tablets (% of drug release, n = 3 tablets)

| 15 min | 30 min | 60 min |
|---|---|---|
| 81.25 | 101.77 | 101.40 |

Dissolution profile of coated tablets (% of drug release, n = 3 tablets)

| 15 min | 30 min | 60 min |
|---|---|---|
| 67.05 | 100.54 | 101.51 |

Dissolution profile parameters
Dissolution media: 0.01N HCl
Volume of test media: 500 ml
Method: Basket method (100 rpm)
PVP K30*polyvinylpyrrolidone with aqueous viscosity of 5.5-8.5 mPas at 10% w/w concentration and 44,000-54,000 average molecular weight

Variant 3: 16 mm extruder, extrudate temeperature 110-120° C.

| VARIANT 3 | mg/unit |
|---|---|
| Internal phase (Melt Extrudate) | |
| Aliskiren hemifumarate | 331.5 |
| PVP K30* | 30.79 |

Melt Extrusion Processing Temp. = 110° C.-Low Shear-16 mm extruder

| | |
|---|---|
| External phase | |
| Polyvinylpyrrolidone XL | 96.4 |
| Microcrystalline cellulose Grn | 207.71 |
| Aerosil 200 | 3.6 |
| Magnesium stearate | 10.0 |
| Total Core tablet weight | 680 mg |
| Mean Hardness [N] | 205-215 |
| Friability [500 drops] | 0% |
| Disintegration time of core tablets | 16-17 min |
| Disintegration time of coated tablets | 19 min |

Dissolution profile of core tablets (% of drug release, n = 3 tablets)

| 15 min | 30 min | 60 min |
|---|---|---|
| 79.43 | 100.56 | 101.43 |

Dissolution profile parameters
Dissolution media: 0.01N HCl
Volume of test media: 500 ml
Method: Basket method (100 rpm)
PVP K30*polyvinylpyrrolidone with aqueous viscosity of 5.5-8.5 mPas at 10% w/w concentration and 44,000-54,000 average molecular weight

### Example 3

### Melt Extrudate: Aliskiren Hemifumarate and HPMC 3cps

16 mm extruder, extrudate temperature 110-120° C.

| | mg/unit |
|---|---|
| Internal phase (Melt Extrudate) | |
| Aliskiren hemifumarate | 331.5 |
| HPMC 3 cps | 24 |

Melt Extrusion Processing Temp. = 110° C.-Low Shear-16 mm extruder

| | |
|---|---|
| External phase | |
| Polyvinylpyrrolidone XL | 96.4 |
| Microcrystalline cellulose Grn | 214.5 |
| Aerosil 200 | 3.6 |
| Magnesium stearate | 10.0 |
| Total Core tablet weight | 680 mg |
| Mean Hardness [N] | 208-229 |
| Friability [500 drops] | 0% |
| Disintegration time of core tablet | 15 min |

### Example 4

### Melt Extrudate: Aliskiren Hemifumarate, PVP K-30 and PVP XL

16 mm extruder, extrudate temperature 105-115° C.

| | mg/unit |
|---|---|
| Internal phase (Melt Extrudate) | |
| Aliskiren hemifumarate | 331.5 |
| PVP K-30* | 15.4 |
| Polyvinylpyrrolidone XL | 15.4 |

Melt Extrusion Processing Temp. = 110° C.-Low Shear-16 mm extruder

| | |
|---|---|
| External phase | |
| Polyvinylpyrrolidone XL | 81.0 |
| Microcrystalline cellulose Grn | 223.1 |
| Aerosil 200 | 3.6 |
| Magnesium stearate | 10.0 |
| Total Core tablet weight | 680 mg |
| Mean Hardness [N] | 196-216 |
| Friability [500 drops] | 0% |
| Disintegration time of core tablet | 20 min |

PVP K30*polyvinylpyrrolidone with aqueous viscosity of 5.5-8.5 mPas at 10% w/w concentration and 44,000-54,000 average molecular weight

### Example 5

### Melt Extrudate: Aliskiren Hemifumarate

Variant 1: 16 mm extruder, extrudate temperature 95-100° C.

| VARIANT 1 | mg/unit |
|---|---|
| Internal phase (Melt Extrudate) | |
| Aliskiren hemifumarate | 331.5 |

Melt Extrusion Processing Temp. = 95° C.; High shear-16 mm extruder

| | |
|---|---|
| External phase | |
| PVP K-30*/HPMC 3 cps | 24 |
| Polyvinylpyrrolidone XL | 96.4 |
| Microcrystalline cellulose Grn | 214.5 |
| Aerosil 200 | 3.6 |
| Magnesium stearate | 10.0 |
| Total Core tablet weight | 680 mg |

PVP K30*polyvinylpyrrolidone with aqueous viscosity of 5.5-8.5 mPas at 10% w/w concentration and 44,000-54,000 average molecular weight

Variant 2: 16 mm extruder, extrudate temperature 95-100° C.

| VARIANT 2 | mg/unit |
|---|---|
| Internal phase (Melt Extrudate) | |
| Aliskiren hemifumarate | 331.5 |

Melt Extrusion Processing Temp. = 70 C.; High shear-16 mm extruder

| | |
|---|---|
| External phase | |
| Polyvinylpyrrolidone XL | 82.36 |
| Microcrystalline cellulose Grn | 154.628 |
| Aerosil 200 | 3.016 |
| Mag stearate | 8.468 |
| Total Core tablet weight | 580 mg |
| Mean Hardness [N] | 170-200 |
| Friability [500 drops] | 0.2% |
| Disintegration time of core tablet | 16 min |

### Example 6

### Melt Extrudate: Aliskiren and HPC

16 mm extruder, extrudate temperature 95-100° C.

| | | Composition per | Composition |
|---|---|---|---|
| | | unit [mg/unit] | per unit [%] |
| Aliskiren | Aliskiren hemifumarate | 331.5 | 48.75 |
| | HPC* | 24.0 | 3.53 |
| | Cellulose MKGR | 214.5 | 31.54 |
| | Crospovidone | 96.4 | 14.18 |
| | Aerosil 200 | 3.6 | 0.53 |
| | Magnesium stearate | 10.0 | 1.47 |
| | Total | 680.0 mg | 100.00 |

| | |
|---|---|
| Mean Hardness [N] | 220 N (170-250) |
| Friability 10St./6.5 g 500 U [%] | 0.1% |
| Disintegration time for Aliskiren [minutes] | 15 min-20 min |

HPC*hydroxypropylcellulose with aqueous viscosity of 300-600 mPas at 10% w/w concentration and 80,000 average molecular weight

### DISSOLUTION TESTING

The dissolution property of the formulations in accordance with the present invention were confirmed by the use of a paddle method at pH 4.5 or 1, as follows. The paddle method assembly consists of the following: a covered vessel made of glass or other inert, transparent material; a motor, and a paddle formed from a blade and shaft as the stirring element. The vessel is partially immersed in a suitable water bath of any convenient size or placed in a heating jacket. The water bath or heating jacket permits holding the temperature inside the vessels at 37±0.5° during the test and keeping the bath fluid in constant, smooth motion. No part of the assembly, including the environment in which the assembly is placed, contributes significant motion, agitation, or vibration beyond that due to the smoothly rotating stirring element. Apparatus that permits observation of the specimen and stirring element during the test is has the following dimensions and capacities: the height is 160 mm to 210 mm and its inside diameter is 98 mm to 106 mm. Its sides are flanged at the top. A fitted cover may be used to retard evaporation.

The shaft is positioned so that its axis is not more than 2 mm at any point from the vertical axis of the vessel and rotates smoothly without significant wobble. The vertical center line of the blade passes through the axis of the shaft so that the bottom of the blade is flush with the bottom of the shaft. The distance of 25±2 mm between the blade and the inside bottom of the vessel is maintained during the test. The metallic or suitably inert, rigid blade and shaft comprise a single entity. A suitable two-part detachable design may be used provided the assembly remains firmly engaged during the test. The paddle blade and shaft may be coated with a suitable inert coating. The dosage unit is allowed to sink to the bottom of the vessel before rotation of the blade is started. A small, loose piece of non-reactive material such as not more than a few turns of wire helix may be attached to dosage units that would otherwise float. Other validated sinker devices may be used.

For Basket Method at pH 6.8:

The assembly consists of the following: a covered vessel made of glass or other inert, transparent material; a motor, a metallic drive shaft; and a cylindrical basket. The vessel is partially immersed in a suitable water bath of any convenient size or placed in a heating jacket. The water bath or heating jacket permits holding the temperature inside the vessels at 37±0.5° during the test and keeping the bath fluid in constant, smooth motion. No part of the assembly, including the environment in which the assembly is placed, contributes significant motion, agitation, or vibration beyond that due to the smoothly rotating stirring element. Apparatus that permits observation of the specimen and stirring element during the test is has the following dimensions and capacities: the height is 160 mm to 210 mm and its inside diameter is 98 mm to 106 mm. Its sides are flanged at the top. A fitted cover may be used to retard evaporation.

The shaft is positioned so that its axis is not more than 2 mm at any point from the vertical axis of the vessel and rotates smoothly without significant wobble. A speed regulating device is used that allows the shaft rotation speed to be selected and maintained at 100 rpm. Shaft and basket components of the stirring element are of stainless steel type 316 or equivalent. The dosage unit is placed in a dry basket at the beginning of each test. The distance between the inside of the bottom of the vessel and the basket is maintained at 25±2 mm during the test.

The Dissolution Medium* (1L unless otherwise indicated) is placed in the vessel of the apparatus. The Dissolution Medium is equilibrated to 37±0.5°, and the thermometer is removed. 1 dosage form (e.g. tablet or capsule) is placed on the apparatus, taking care to exclude air bubbles from the surface of the dosage-form unit, and immediately the apparatus is operated at a rate of 75±3 rpm or 100±3rpm depending on the pH. Within the time interval specified (e.g. 10, 20, 30, 45, 60, 90 and 120 min.), or at each of the times stated, a specimen(≧1 ml) is withdrawn from a zone midway between the surface of the Dissolution Medium and the top of the rotating blade, not less than 1 cm from the vessel wall. [NOTE—the aliquots withdrawn for analysis are replaced with equal volumes of fresh Dissolution Mediums at 37° or, where it can be shown that replacement of the medium is not necessary, the volume change is corrected in the calculation. The vessel is kept covered for the duration of the test, and the temperature of the mixture under test at suitable times is verified.]. The specimen is filtered through a suitable filter, e.g. a 0.45 μm PVDF filter (Millipore) and the first mls (2 to 3 ml) of the filtrate are discarded. The analysis is performed by HPLC or UV detection. The test is repeated at least 6 times with additional dosage form units.

Dissolution medium for pH 4.5: 1L of a buffered aqueous solution, adjusted to pH 4.5±0.05 (0.1 M Phosphate buffer solution obtained by dissolving 13.61 g of potassium hydrogen phosphate in 750 ml of deionized water and diluted to 1L with deionized water)

Dissolution medium for pH 1: 500 ml, unless otherwise indicated, of 0.01M hydrogen chloride.

Dissolution medium for pH 6.8: 1L of a buffered aqueous solution, adjusted to pH 6.8±0.05 (0.05 M phosphate buffer solution obtained by dissolving 6.8 g of potassium hydrogen phosphate and 0.9 g sodium hydroxide in 1L deionized water).

What is claimed is:

1. A solid oral dosage form comprising aliskiren, or a pharmaceutically acceptable salt thereof, alone or in combination with another active agent, wherein aliskiren, or a pharmaceutically acceptable salt thereof, is obtainable by melt granulation with optionally one or more granulation excipient.

2. A solid oral dosage according to claim 1, wherein aliskiren is present in an amount of 20% or more by weight based on the total weight of the oral dosage form.

3. A solid oral dosage form according to claim 1, wherein aliskiren is present in an amount ranging of from 20 to 80%, such as of from 30 to 70%, such as of from 40 to 60%, in particular of from 40 to 55%, such as of from 44 to 52%, by weight based on the total weight of the oral dosage form.

4. A solid oral dosage form according to any of the preceding claims, wherein aliskiren, or a pharmaceutically acceptable salt thereof, is present in an amount ranging of from 75 to 300 mg of the free base per unit dosage form.

5. A solid oral dosage form according to any of the preceding claims, wherein Aliskiren is in the form of a hemi-fumarate thereof, and is present in an amount of about 83, about 166 or about 332 mg per unit dosage form.

6. A solid oral dosage form according to any of the preceding claims, wherein aliskiren is obtainable by melt granulation with one or more polymers.

7. A solid oral dosage form according to claim 6, wherein the polymer is a polymer of of N-vinyl pyrrrolidone or of cellulose.

8. A solid oral dosage form according to claim 7, wherein the polymer is selected from hydroxypropylmethylcellulose, hydroxypropylcellulose and polyvinylpyrrolidone.

9. A solid oral dosage form according to claim 6, wherein the polymer is hydroxypropylcellulose.

10. A solid oral dosage form according to any one of claims 6 to 9, wherein the ratio of aliskiren to polymer is of from 80:20 to 98:2, preferably of from 85:15 to 96:4.

11. A solid oral dosage form according to any of the preceding claims, wherein aliskiren is present in an amount ranging of from 60 to 100%, such as of from 75 to 100%, such as of from 80 to 100%, for example of from 85% to 100%, by weight based on the total weight of the granulate obtainable by melt granulation.

12. A solid oral dosage form according to any of the preceding claims, wherein the dosage form further comprises a filler, preferably in an amount of 12 to 45% by weight of the dosage form.

13. A solid oral dosage form according to claim 12, wherein the filler is microcrystalline cellulose.

14. A solid oral dosage form according to any of the preceding claims, wherein the dosage form further comprises a disintegrant, preferably in an amount of 5 to 30% by weight of the dosage form.

15. A solid oral dosage form according to claim 14, wherein the disintegrant is crospovidone.

16. A solid oral dosage form according to any of the preceding claims, wherein the dosage form further comprises a lubricant, preferably in an amount of 0.5 to 4% by weight of the dosage form.

17. A solid oral dosage form according to claim 16, wherein the lubricant is magnesium stearate.

18. A solid oral dosage form according to any of the preceding claims, wherein the dosage form further comprises a glidant, preferably in an amount of 0.1 to 1.0% by weight of the dosage form.

19. A solid oral dosage form according to claim 18, wherein the glidant is aerosil 200.

20. A solid oral dosage form according to any of the preceding claims, wherein the dosage form further comprises valsartan, or a pharmaceutically acceptable salt thereof.

21. A solid oral dosage form according to claim 20, wherein aliskiren, or a pharmaceutically acceptable salt thereof, is physically separated from valsartan, or a pharmaceutically acceptable salt thereof.

22. A solid oral dosage form according to claim 20 or 21, in the form of a bilayer tablet comprising a layer comprising aliskiren, or a pharmaceutically acceptable salt thereof, and a layer comprising valsartan, or a pharmaceutically acceptable salt thereof.

23. A solid oral dosage form according to any one of claims 20 to 22, wherein valsartan, or a pharmaceutically acceptable salt thereof, is obtainable in the form of a granulate by roller compaction.

24. A solid oral dosage form according to any one of claims 20 to 23, wherein valsartan, or a pharmaceutically acceptable salt thereof, is present in an amount ranging of from 75 to 350 mg of the free acid per unit dosage form.

25. A solid oral dosage form according to any of the preceding claims for the treatment of hypertension, congestive heart failure, angina, myocardial infarction, artherosclerosis, diabetic nephropathy, diabetic cardiac myopathy, renal insufficiency, peripheral vascular disease, left ventricular hypertrophy, cognitive dysfunction, stroke, headache and chronic heart failure.

26. A method for the treatment of hypertension, congestive heart failure, angina, myocardial infarction, artherosclerosis, diabetic nephropathy, diabetic cardiac myopathy, renal insufficiency, peripheral vascular disease, left ventricular hypertrophy, cognitive dysfunction, stroke, headache and chronic heart failure which method comprises administering a therapeutically effective amount of a solid oral dosage form according to any of claims 1 to 11 to a patient in need thereof.

27. Use of a solid oral dosage form according to any of claims 1 to 25 for the manufacture of a medicament for the treatment of hypertension, congestive heart failure, angina, myocardial infarction, artherosclerosis, diabetic nephropathy, diabetic cardiac myopathy, renal insufficiency, peripheral vascular disease, left ventricular hypertrophy, cognitive dysfunction, stroke, headache and chronic heart failure.

28. A process for the manufacture of a solid oral dosage form according to any of the preceding claims comprising the steps of melt extruding Aliskiren or a pharmaceutical acceptable salt thereof and optionally one or more granulation excipients; optionally mixing with pharmaceutically acceptable additives; and optionally compressing the final blend into a tablet.

29. A process according to claim 28 further comprising the step of adding the granulate of a further active ingredient, for example valsartan, before compression into a tablet.

30. A process for the manufacture of a solid oral dosage form according to any of the preceding claims comprising the steps of:
(a′) melt extruding Aliskiren, or a pharmaceutical acceptable salt thereof, and optionally one or more granulation excipients, to form an Aliskiren granulate;
(b′) granulating a further active ingredient, for example valsartan;
(c′) sieving the respective granulates;
(d′) optionally mixing the respective granulates with outer phase excipients; and
(e′) compressing both granulates together to form a bilayer.

31. A process according to anyone of claims 28 to 30 comprising the steps of;
(a) blending Aliskiren or a pharmaceutical acceptable salt thereof, and optionally one or more granulation excipients to give a preblended material;
(b) sieving the preblended material to give a screened material;
(c) blending the sieved material to give a blended material;
(d) melt extruding the blended material to give an extrudate;
(c) cooling the extrudate;
(d) milling the cooled extrudate;
(e) optionally blending the milled extrudate with one or more pharmaceutically acceptable excipients to form a final blended material;
(f) optionally compressing the final blend to form a tablet; and
(g) optionally applying a film coat in order to obtain a film coated tablet.

32. A process according to claims 31, wherein the temperature of the extrudate (step d) is of 95° C. or higher, for example 100° C. or higher, such as 110° C. or higher, for example of from 95° C. to 130° C., in particular of from 100° C. to 120° C., such as of from 105° C. to 115° C.

33. A process according to any of claims 30 to 32, wherein the entire process is a continuous process.
